(19) United States
(12) Patent Application Publication
(10) Pub. No.: US 2006/0276684 A1
(43) Pub. Date: Dec. 7, 2006

(54) Device and method for treating congestive heart failure

(76) Inventor: Giovanni Speziali, Rochester, MN (US)

Correspondence Address: QUARLES & BRADY LLP, FIRSTAR PLAZA, ONE SOUTH PINCKNEY STREET, P.O BOX 2113 SUITE 600, MADISON, WI, 53701-2113, US

(21) Appl. No.: 10/572,112

(22) PCT Filed: Nov. 5, 2004
(86) PCT No.: PCT/US04/37221

§ 371 Date: Mar. 16, 2006

Related U.S. Application Data
(60) Provisional application No. 60/518,531, filed on Nov. 7, 2003.

(51) Int. Cl. A61F 2/00

(52) U.S. Cl. 600/37; 600/16

(57) ABSTRACT

A method for treating congestive heart failure in a minimally invasive manner. The method employs a thoracoscopic device which is inserted through a patient's chest wall and into the beating heart. The device carries an elastic containment system which is transported through a lumen of the device and means for deployment of such a containment system within a chamber of the heart. The elastic containment system employs en elastic suture with at least one end anchored in the chamber wall by attached non-retraceable needle. When deployed, the elastic suture produces an increasing tensile force as the chamber expands that assists during subsequent contraction.

### CROSS-REFERENCE TO RELATED APPLICATIONS

This application claims the benefit of U.S. Provisional Application No. 60/518,531 filed on Nov. 7, 2003, which is hereby incorporated by reference herein.

### BACKGROUND OF THE INVENTION

In general, dilatation of the left ventricular cavity is closely correlated with decline of the left ventricular function and development of congestive heart failure (CHF). In the past multiple attempts at surgical correction of left ventricular dilatation have been carried out internationally with the purpose of delaying the progression to intractable CHF. These attempts have included a variety of invasive procedures such as for example, left ventricular volume reduction techniques (e.g., Batista Operation, Dor Procedure, and resectioning of left ventricular aneurysm) and left ventricular “containment” techniques. The implantation of various “containment systems” which are believed to be invasive, have been shown to help prevent left ventricular dilatation and perhaps even delay the decline in ventricular function leading to CHF. Such systems include for example, the MyoSplint, ACORN Net, and undersized mitral annuloplasty.

Furthermore, a number of methods and devices have been recently proposed in the literature in order to increase the contractile capacity of the cardiac muscle, limit diastolic volume and reduce cardiac wall stress. For example, U.S. Pat. No. 5,192,314 describes an apical cap inserted into the ventricle; however, the cap does not allow a reduction in equatorial diameter and fails to reach the objective of restoring the optimal geometry of the ventricle.

Patent application No. WO9944534 describes epicardial bands whose drawback is that they may interfere with diastolic function insofar as they may cause greater volumetric constriction. Furthermore, these bands make up a static device and do not allow the restoration of optimal ventricular geometry.

Patent application WO0006027 also described a ring, not attached either to the ventricular wall or to the mitral anulus, that is rigid enough to hold the submitral apparatus with the only purpose of being a restrictive device.

In U.S. Pat. No. 5,674,280 a valvular annuloplasty ring is described whose main characteristic is that of being fabricated from a low elasticity metal alloy and therefore with no possible direct activity on ventricular function.

More recently, U.S. Pub. App. No. 20030158570 reported using endoventicular devices for the treatment and correction of cardiomyopathies. This application discloses a device having elastic elements in the radial direction towards the inside of the ventricle and plastic deformation in a direction that is transversal to the ventricle (e.g., ring-like suture). The ring-like suture is then attached to the internal wall of the ventricle. However, as with all of the earlier devices, they were inserted into the ventricle by performing invasive open-heart surgery.

There have also been a series of patents assigned to Myocor, Inc. (St. Paul, Minn.) which disclose various devices for treatment of a failing heart by reducing the wall tension therein. These devices generally include a tension (elastic) member for drawing at least two walls of a heart chamber toward each other. The common theme is the deployment in many different patterns of elastic members that extend across one side of the heart to the other and are anchored by pads or hinged anchors that rest against the outside of the chamber wall. In order to practice this procedure, it requires opening the chest cavity and performing open heart surgery, which is quite invasive. There is no indication that these elastic support structures can be erected from inside the left ventricle chamber using any type of catheter or non-invasive mechanism.

Other recent approaches for supporting the heart wall have included use of an exterior constraining device, such as those disclosed in U.S. Pat. Nos. 5,702,343 and 6,165,122. These patents disclose a cardiac reinforcement device (CRD) for the treating cardiomyopathies. Essentially, the CRD is a mesh-like material covering the heart like a jacket or a girdle and provides reinforcement of the heart walls by constraining cardiac expansion, beyond a predetermined limit.

Furthermore, there have been attempts to perform cardiac procedures without opening the chest cavity. Typically, minimally invasive procedures are conducted by inserting surgical instruments and an endoscope or thoracoscope through small incision in the skin of the patient. In these procedures manipulating such instruments has proved to be awkward. It has been found that a high level of dexterity is required to accurately control the instruments. Furthermore, it is understood that these procedure have been performed by stopping the hear, which clearly adds an additional risk factor to cardiac surgery.

There have been other methods and devices for performing minimally invasive surgical procedures, such as that described in U.S. Pat. No. 6,063,095. These devices and methods would include endoscopic coronary artery bypass graft (E-CABG) and other anastomotic procedures. It is believed that currently, even with hand positioned instruments, the precision necessary for such suturing is lacking. Also, none of these procedures is performed in a completely endoscopic manner without stopping the heart.

As such, what is needed in the art is a device and method for performing minimally invasive microsurgery and more particularly endoscopic heart surgery without stopping the heart. Accordingly, less invasive devices and procedures are needed in the industry to help reduce the progression of congestive heart failure.

### SUMMARY OF THE INVENTION

The present invention is a device and method for treatment of congestive heart failure by implanting an elastic containment system inside the heart chamber in a minimally invasive manner. The device includes a thorascope having a distal end which is inserted into the heart chamber; an elastic containment system carried by the distal end; and means for deploying the elastic containment system into the heart chamber by attaching the ends of a suture to the chamber wall. The sutures in the elastic containment system provide a tensile force, which pulls the ventricular walls inward facilitating normal cardiac function.

The elastic containment system can take many forms. The elastic containment system is made of an elastic suture and a plurality of non-retractable needles at its ends. In a first preferred embodiment the suture is a bi-directional suture with needles at both ends. In another embodiment the device provides for a plurality of uni-directional sutures attached to an elastic ring for a radially directed containment system.

Another aspect of the invention is a method for implanting an elastic containment system in a heart chamber. The method includes inserting the intra-cardiac end of a device through the subject's chest wall and into the heart chamber, wherein the device includes a thorascope having a distal end which carries an elastic containment system and means for deploying the elastic containment system into the heart chamber. The elastic containment system is deployed into the heart chamber by attaching the ends of a suture to the chamber wall. This method is practiced without stopping the heart.

A general object of the present invention is to deploy an elastic containment system in the chamber of a beating heart. Without opening the patient's chest and bypassing the heart, the elastic containment system housed in the device is transported into the heart chamber and deployed by the device as described herein. The elastic containment system has needles on the ends of an elastic suture which enable the ends to be attached to the beating heart wall from within the chamber.

The foregoing and other objects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a view in cross-section through the chest of a patient undergoing a procedure according to a preferred embodiment of the invention;

FIG. 2 is a pictorial representation of a patient's heart during the procedure of FIG. 1;

FIG. 3 is a partial view of a patient's heart illustrating axial deployment of preferred embodiments of an elastic containment system which forms part of the present invention;

FIG. 4 is a partial view of a patient's heart illustrating radial deployment of elastic containment systems;

FIG. 5 is a pictorial view of an alternative embodiment of the elastic containment system;

FIG. 6 is a partial view of a patient's heart illustrating deployment of the elastic containment system of FIG. 5;

FIG. 7 is a partial view of a patient's heart illustrating axial deployment of a plurality of elastic containment systems of FIG. 5;

FIG. 8 is a perspective view of the distal end of a thoracoscopic device shown in FIG. 2;

FIG. 9A is a perspective view of a preferred embodiment of a non-retractable needle used in the containment systems of FIGS. 5 and 6;

FIG. 9B is a view in cross-section with parts shown in whole of the non-retractable needle of FIG. 9A disposed on the end of an arm which forms part o the structure in FIG. 8; and

FIG. 9C is a partial view in cross-section of a cardiac wall with a deployed anchor that forms part of the non-retractable needle of FIG. 9A.

### DETAILED DESCRIPTION OF THE PREFERRED EMBODIMENT

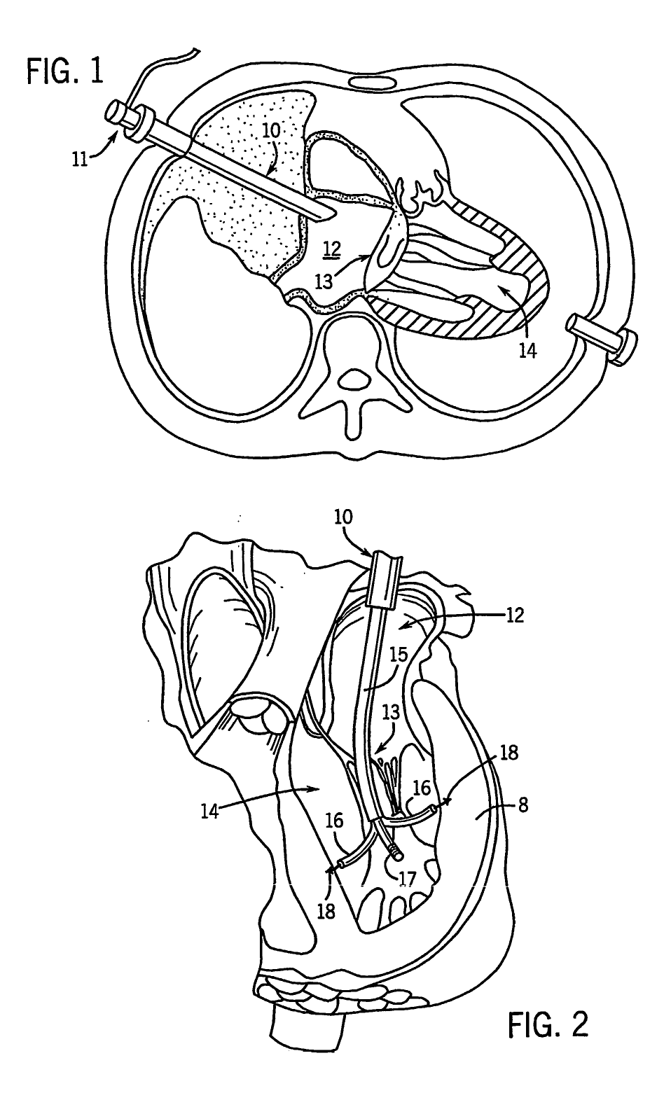

The present invention relates to a device and method for treating congestive heart failure by implanting an elastic containment system inside a beating heart chamber. Referring particularly to FIG. 1, under general anesthesia and double-lumen endotracheal ventilation, the patient is prepped and draped to enable surgical access to the right lateral, anterior and left lateral chest wall. The patient is fully heparinized, and after collapsing the right lung, the pericardium is opened longitudinally and an introducer 10 is inserted in the right chest through intercostal spaces. The introducer 10 has a side port and a hemostatic valve at its proximal end 11 and its distal end is inserted into the left atrium 12 of the patient's heart. The distal end of the introducer 10 faces the plane of the mitral valve 13 that leads to the left ventricle 14. The side port at the proximal end 11 of the introducer is connected to a suction system and blood is continuously and slowly aspirated from the left atrium. This prevents air embolisms inside the cardiac chambers. Aspirated blood is continuously reinfused into the patient.

Referring particularly to FIGS. 1 and 2, the introducer 10 provides access to the interior of the beating heart for a thorascope 15. The thorascope 15 is a long, thin flexible instrument having a diameter of from ⅛ to ½ inches which is inserted through the introducer 10 into the patient's beating heart. The extra-thoracic, proximal end of the thorascope 15 contains a handle (not shown) which enables the physician to manipulate the intra-cardiac distal end of the thoracoscopic device which carries the elastic containment system and the means for deploying such a system. The thorascopic device contains lumens which extend from its proximal to distal ends to enable the elastic containment system described below to be readily transported and be deployed in the patient's beating heart. To practice the present invention the distal end of the thorascopic device is manipulated through the mitral valve 13 and into the left ventricle 14.

Referring particularly to FIG. 2 one device deployed by the thoracoscopic device is an echo probe 17 which can be separately manipulated to view selected subjects within the left ventricle. The echo probe 17 is typically an elongated probe which extends from a proximal end to a distal end of the elongated body. The echo probe 17 contains an ultrasonic transducer on its distal end which enables it to acquire data from which images of the interior of the left ventricle 14 can be reconstructed. These images reveal the anatomical structure of the left ventricle myocardium 8, and by marking the other devices used in the procedure with reflective materials, the locations and movement of those devices can also be observed in real-time by the physician. The echo probe 17 is a catheter-like device such as that disclosed in U.S. Pat. No. 6,129,672 entitled “Volumetric Image Ultrasound Transducer Underfluid Catheter System” which is incorporated herein by reference.

Also a means for deploying an elastic containment system into the heart chamber is delivered by the thoracoscopic device. In the preferred embodiment this deployment device includes a pair of catheter-like devices 16 that extend out the distal end of the thoracoscopic device and which can be separately manipulated by the physician to selected locations on the heart chamber wall 8. These arms 16 carry barbed needles 18 which form part of the elastic containment system, and by manipulating each arm 16, the physician embeds the needles 18 into the myocardium 8 at selected locations in the left ventricle 14. As will be described in more detail below, various types of elastic containment systems may be used, but they may all be deployed from inside the cardiac chamber 14 by manipulating the arms 16. Specifically, the arms 16 may be adjusted to a selected location by for example, rotating the hemostatic valve at the proximal end 11 of the introducer 10. The needles 18 are clearly visible in ultrasonic images which enables the physician to see where they are inserted and how deeply they are inserted into the left ventricle. Each arm 16 forms part of a catheter-like device such as that disclosed in U.S. Pat. No. 6,056,760 entitled “Device For Intracardiac Suture” which is incorporated herein by reference.

Referring particularly to FIG. 8, each arm 16 is the distal end of a flexible shaft 30 which extends through the lumen formed in the thorascopic device to its proximal, extra-thoracic end. The arms 16 are easily bendable and are capable of curving outwards to approximately a ninety degree angle relative to the thorascope when they are pushed out through the distal end of the thorascope 15. The arms 16 can be swung radially around the left ventricle long axis 32 by rotating their respective shafts 30. The axial location of the arms 16 is controlled by moving the distal end of the thoracoscopic device along the long axis 32. Thus, by rotating the two shafts 30 and extending or retracting the thorascope 15, the tips of each arm 16 can be manipulated into contact with the chamber wall 8 at the prescribed locations. Bands of reflective material 34 are painted on the tips of the arms 16 such that they appear brightly in the ultrasound images acquired by echo probe 17.

Other guidance methods can be used during the procedure. For example, transesophageal echocardiography may be employed during placement of the introducer 10 and deployment of the thorascope 15 into the left ventricle.

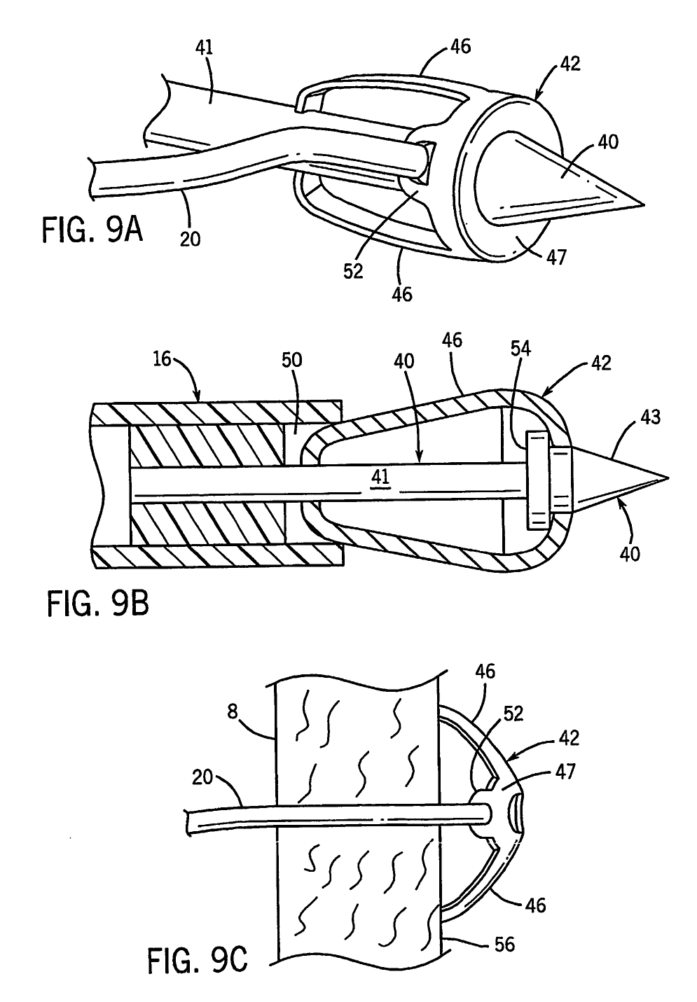

Referring particularly to FIGS. 9A-C, a preferred embodiment of the non-retractable needle 18 includes a metal needle 40 having a pointed distal end 43 and a shank 41 which extends into an opening in the distal end of the arm 16. The metal needle 40 carries a metal anchor 42 having a central opening through which the pointed end 43 extends. The anchor 42 has two arms 46 which extend radially outward from a hub 47 and fold backward along the shank 41 of the needle 40. The ends of the arms 46 are trapped in an annular chamber 50 formed in the end of the arm 16. An eyelet 52 is formed on the hub of the anchor 42 and one end of an elastic suture 20 is fastened to it.

As indicated above, the non-retractable needle 18 is forced completely through the heart wall 8 by applying an axial force to the arm 16. This axial force is conveyed to the needle 40 and to the anchor 42 by a flange 54 formed on the shank 41. The arm 16 is then withdrawn. When withdrawn, the arms 46 on the anchor 42 are freed and spring radially outward and bear against the outer surface 56 of the heart wall. The needle 40 is withdrawn from the anchor 42 as the arm 16 is pulled back.

After the elastic containment system(s) has been deployed, the intra-cardiac end of the above-described thorascopic device is withdrawn, as is the introducer 10 and the atrial wall incision is repaired by either a prepositioned purse-string suture or other hemostatic device or technique. Hemostasis is checked, all thorascopic ports are withdrawn, appropriate chest drainage tubes are positioned and secured, and all thorascopic incisions are closed.

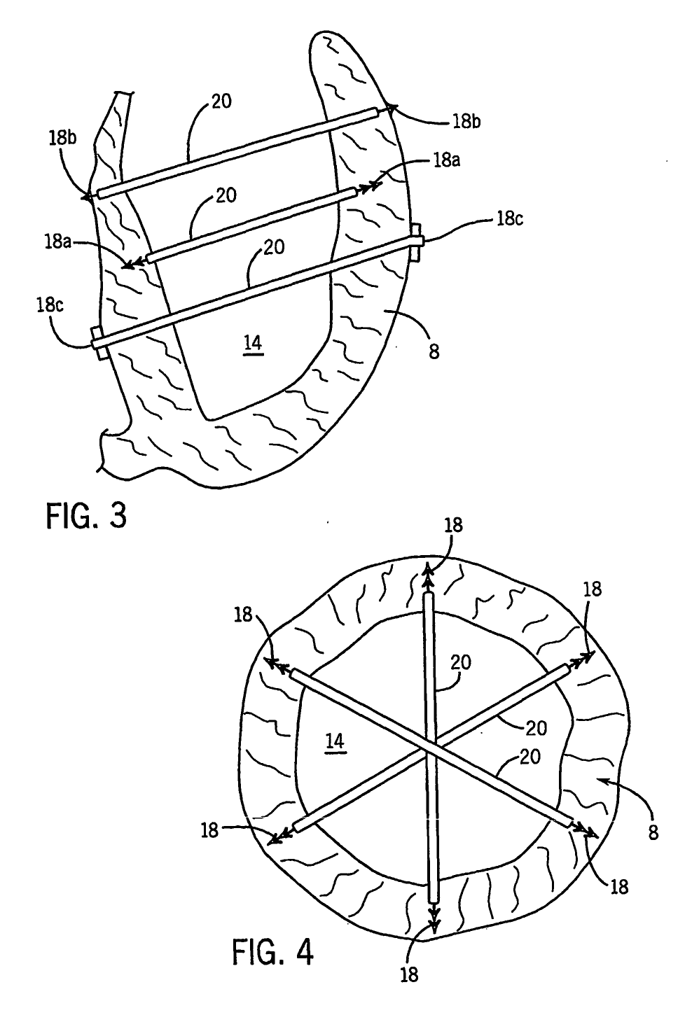

Referring particularly to FIG. 3, in its simplist form the elastic containment system is comprised of an elastic suture 20 with a non-retractable needle 18 connected to each of its ends. A flexible polymer material may be used and its elasticity will depend on the prescribed force needed to properly contract the ventricle wall 8. The non-retractable needles can take a number of different forms. In one embodiment the needles 18a are harpoon, or barbed shaped and are embedded inside the myocardium 8.

In another embodiment the needles 18b are pushed completely through the heart wall 8 and the needle 18b expands radially outward to prevent it from being withdrawn. In a third embodiment the needles 18c are straight and the suture end 20 is attached at mid-length of the needle 18c. When the needle 18c is pushed completely through the heart wall 8, it pivots 90° about this connection point to prevent withdrawal. Also, another approach for deploying a suturing anchor is described in U.S. Pat. No. 6,719,767 incorporated herein by reference. This patent discloses a suturing means having a type of “clip” which has two arms pivotally connected to each other used to capture pre-selected sections of the cardiac walls.

The force produced by the single-suture elastic containment system of FIG. 3 is along a single axis. This bi-directional force can be applied in any direction by judicious placement of the two needles 18 in the heart wall 8. As shown in FIG. 4, by deploying a plurality of the bi-directional elastic containment systems in different radial directions, forces can be applied around the entire circumference of the heart chamber 14. These can be deployed in substantially the same plane to emulate a band of elastic material disposed around the circumference of the heart, or they can be deployed along the long axis of the left ventricle as shown in FIG. 3.

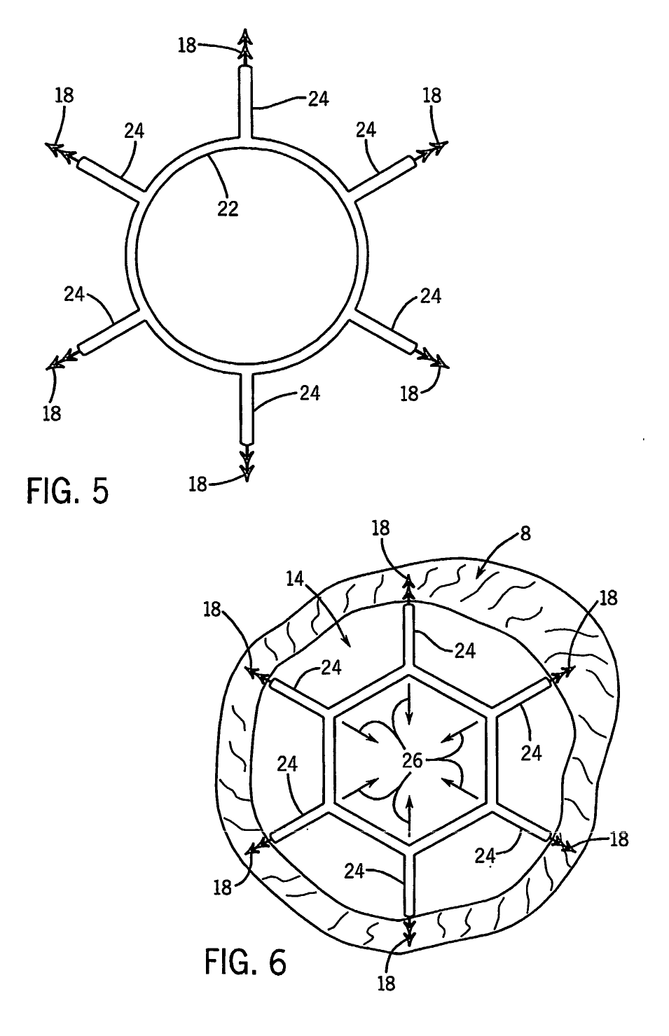

When it is desired to produce radially directed forces in substantially the same plane, an alternative embodiment of the elastic containment system is deployed. Referring particularly to FIGS. 5 and 6, this radial elastic containment system includes an elastic ring 22 and a plurality of radial elastic sutures 24. In the preferred embodiment there are six radial sutures 24 spaced equally around the elastic ring 22. It should be apparent, however, that the number and location of the radial sutures 24 can be varied to mold the pattern of radially directed contraction forces to meet the prescribed clinical needs. Non-retractable needles 18 are fastened to the end of each radial suture 24 and these are embedded in the heart chamber wall 8 as described above.

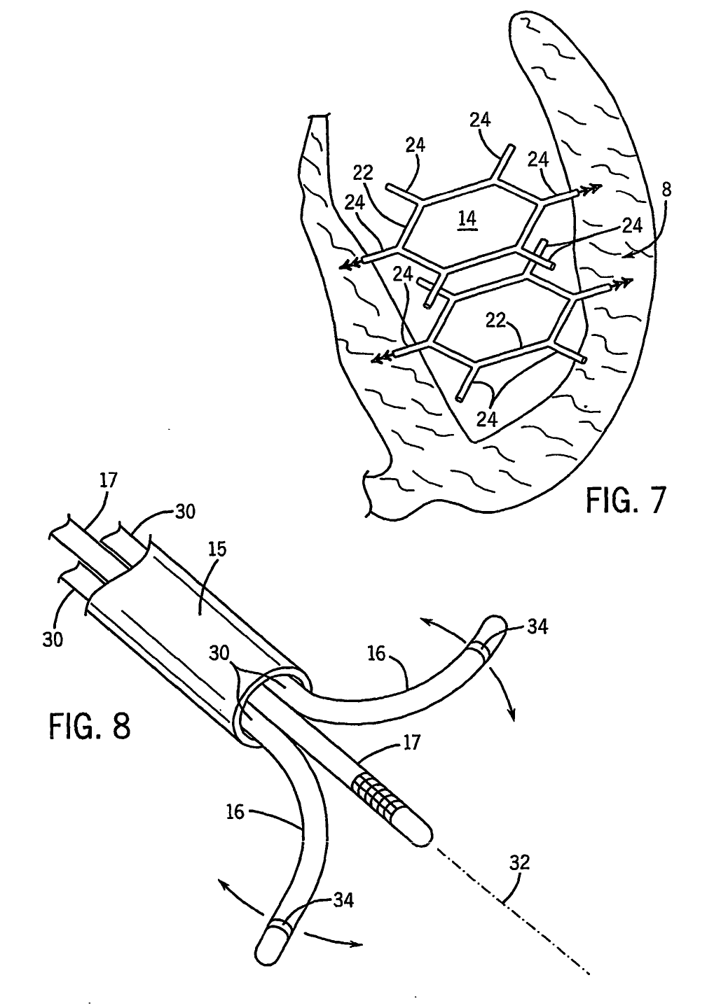

When deployed in a plane as shown in FIG. 6, forces directed radially inwards as indicated by arrows 26 are produced by this device as the heart wall expands outward. These forces 26 increase non-linearly as the heart expands to provide contracting forces that supplement the contractile forces produced by the heart wall. As shown in FIG. 7, a plurality of radial elastic containment systems may be deployed at different locations along the left ventricle long axis to enable the supplemental contracting forces 26 to be produced in a prescribed pattern along the axial extent of the left ventricle.

The containment system is designed such that it allows multiple and modular distribution of the sutures to facilitate contraction of the left ventricular muscle in a prescribed manner. The elastic containment system may be broadly described as a resilient (having the characteristic of being elastically deformable) endocardial device designed to reduce one or more diameters, as well as the volume of the heart chamber, by reducing its mitral annulus and/or equatorial circumference and/or apex. Furthermore, the containment system is characterized by elastic properties having non-linear elasticity, which allows the elastic containment system to act as an aid to systolic function during the contraction phase. With respect to the diastolic function, the same nonlinear elasticity means that the device does not interfere with diastolic function: in fact, although opposing a progressively increasing resistance against dilatation, the containment system does not statically constrict the heart by impeding its expansion within physiological limits, as in the case of the devices described in WO9814136 and WO9944534.

Each of the individual components of the embodiments described above should be formed from suitable biocompatible materials known to those skilled in the art and may have such dimensions as to be readily introducible into the left ventricle. Typical materials can include, among other biocompatible materials, stainless steel, titanium alloys, NiTi alloys such as Nitinol or elgiloy. Preferably, the elastic suture 20 with a non-retractable needle 18 connected to at least one end is composed of a memory material such as Nitinol. As a result, the elastic suture 20 with a non-retractable needle 18 can easily be made to transform from a substantially closed state as they are transported from the proximal end through to the distal end of the thoracoscopic device to an open state when the elastic suture 20 with the non-retractable needle 18 are suitably deployed to reach and capture to the left ventricle walls.

In order to reduce biological attack on the individual members of the elastic containment system, suitable drugs can be incorporated into a binder coating. Suitable drugs include heparin, Coumadin, anti-inflammatory steroid or ASA-aspirin. The oxide layer of the underlying metal can also be optimized to improve bio-resistance. This is particularly true for stainless steel, titanium, or nickel titanium on which an oxide layer can be formed by heating the component to improve biocompatibility. Further coatings include calcium hydroxy appetite, beta tricalcium phosphate and aluminum oxide can be applied to the members of the elastic containment system.

The elements of the elastic containment system are preferably echo cardiographic compatible, or includes a marker (e.g., biocompatible metal) which is echo compatible. This feature of the invention is particularly desirable for follow-up, non-invasive monitoring of the elements of the elastic containment system after implantation. The preferred locations for markers include the center of the elastic sutures and at the anchor points in the heart wall. The presence of the elements of the containment system can be visualized using an ultrasound imaging device and the distance between two or more markers measured. Integrity of the elements of the containment system can be confirmed as well.

It is also possible to incorporate sensing devices into the elastic containment system. For example, a strain gauge can be integrated into an elastic suture to monitor the force which it produces during a cardiac cycle. The strain gauge can be connected by biocompatible leads to a conventional monitoring device or radio frequency communication can be employed.

Also, in relation to other less invasive bodily locations from which the device may be introduced, applicants envision that the intra-cardiac end of the above-described device carrying the elastic containment system and means for deploying the elastic containment system may be assembled on the extremity of a flexible catheter, and may be introduced into the heart chamber percutaneously. For example, an alternative route or entry into the left ventricle may be through making a percutaneous incision in a patient's artery, such as the femoral artery, in a manner similar to a percutaneous transluminal coronary angioplasty. However, in the present procedure, the distal end of the catheter with the deployment system could be advanced through the arterial system (e.g., femoral artery or brachial artery) and is passed in a retrograde fashion to the blood flow through the aorta and through the aortic valve ultimately reaching the left ventricle cavity. This percutaneous embodiment is generally described by U.S. Pat. No. 6,719,767 entitled “Device and a method for treatment of atrioventricular regurgitation” which is incorporated herein by reference.

In another embodiment, the catheter may be introduced into a vein and passed up to the heart via the vein. The catheter could be introduced into the left ventricle through any suitable vein, such as the femoral, jugular or subclavian veins of the venous system. In this embodiment, the catheter preferably passes through the interatrial septum to the left atrium and then passed through the mitral valve until reaching the left ventricle cavity. Then, once the extremity of the device containing the deployment mechanism has reached the left ventricle, at this point the sutures of the elastic containment system would be deployed and anchored in the wall of the left ventricle cavity. It is believed that this approach would be an even less invasive procedure for the deployment of the elastic containment system into the heart chamber, which could substantially reducing trauma, risk of complication, recovery time, and pain for the patient.

Applicants also envision the possibility of combining the implantation of this elastic containment system with other epicardial and intracardiac procedures (mitral valvuloplasty, mitral valve replacement, aortic valve replacement, CABG, etc) made necessary by the disease, and it is likewise possible to personalize the ventricular remodeling on the basis of the functional. volumetric and geometric characteristics of the patient's ventricle by using the containment system in different ways (in different numbers and sizes).

One of the advantages of the method of the invention is that smaller and less invasive incisions may be used during the surgical procedure. This method eliminates the need for sternotomy (cutting the chest and pulling the ribs apart to gain access to the heart), reducing trauma and facilitating quicker recovery. The method is also easily adapted to meet the specific needs of each patient. The length of elastic sutures can be determined based upon the size and condition of the patient's heart.

While the method and apparatus described herein is particularly suited for use with the left ventricle of the heart, it is contemplated that the elastic containment system may also be deployed in other heart chambers.

1. A method for deploying an elastic containment system in a chamber of a beating heart of a subject, the steps comprising:
a) inserting a distal end of a thoracoscopic device through the subject's chest wall and into the heart chamber; or
b) optionally, inserting the distal end of the thorascopic device percutaneously through the subject's arterial system or venous system, and into the heart chamber;
c) transporting the elastic containment system through a lumen in the thoracoscopic device to the distal end in the heart chamber; and
d) deploying the elastic containment system by anchoring ends of an elastic suture on the elastic containment system in the wall of the heart chamber by penetrating the wall from within the chamber.

2. The method as recited in claim 1 in which step a) includes passing an introducer through the chest wall and through an incision in the heart, and passing the thorascope through the introducer and into the heart.

3. The method as recited in claim 2 in which the chamber is the left ventricle, the incision is in the wall of the left atrium, and the thoracoscopic device passes through the left atrium and its distal end is located in the left ventricle.

4. The method as recited in claim 1 in which the elastic containment system has non-retractable needles on the ends of the suture and step d) includes pushing the non-retractable needles into the heart wall.

5. The method as recited in claim 4 in which the non-retractable needles are pushed through the heart wall and engage the outer surface of the heart wall.

6. The method as recited in claim 1 in which steps c) and d) are repeated to deploy a plurality of elastic containment systems in said heart chamber.

7. An elastic containment system for deployment from within the chamber of a patient's heart, the combination comprising:
an elastic suture having a plurality of ends, the elastic suture producing a force which resists movement of the ends away from each other; and
a plurality of non-retractable needles, each connected to respective ends of said elastic suture, each non-retractable needle being shaped to facilitate penetration of the chamber wall from within the chamber and to inhibit withdrawal of the needle from the chamber wall, and thereby anchor it in place;
wherein the elastic suture produces a force pulling the anchored non-retractable needles towards one another when the chamber wall in which they are anchored expands.

8. The elastic containment system as recited in claim 7 in which the elastic suture is substantially straight and has two ends, with a non-retractable needle attached to each end.

9. The elastic containment system as recited in claim 7 in which the elastic suture includes a ring and a plurality of radially directed elastic suture elements connect to the ring at locations around its circumference, each elastic suture element having an end fastened to a non-retractable needle.

10. The elastic containment system as recited in claim 9 in which the ring is constructed of an elastic material.

11. A system for treating congestive heart failure in a subject, the combination comprising:
a thorascope having a distal end for insertion through the subject's chest wall and into a chamber of the subjects heart, the thorascope having a lumen therein extending from its distal end to a proximal end;
an elastic containment system having an elastic suture with a plurality of ends and non-retractable needles attached to the respective ends; and
means for transporting the elastic containment system in the lumen to the distal end of the thorascope and for deploying the elastic containment system in the chamber by anchoring said non-retractable needles in the wall of said chamber.

12. The system as recited in claim 11 which includes an echo probe extending through the lumen and having a transducer mounted at its distal end and extending into the chamber for acquiring ultrasound data from which an image of the chamber wall and said elastic containment system is reconstructed.
